(19) United States
(12) Patent Application Publication
(10) Pub. No.: US 2015/0343148 A1
(43) Pub. Date: Dec. 3, 2015

(54) DEVICE COMPRISING A BODY, A PUSHER, AND A CAP, THE CAP AND/OR THE BODY COMPRISING A HYDROPHILIC TREATMENT

(71) Applicant: ASSOCIATION POUR LES TRANSFERTS DE TECHNOLOGIES DU MANS, LE MANS (FR)

(72) Inventor: Pascal MARMEY, Le Mans (FR)

(21) Appl. No.: 14/410,205

(22) PCT Filed: Jun. 27, 2013
(86) PCT No.: PCT/IB2013/055283

§ 371 Date: Dec. 22, 2014

(30) Foreign Application Priority Data
Jun. 27, 2012 (FR) 1256128

(51) Int. Cl. A61M 5/315; A61B 5/153; A61B 5/15

(52) U.S. Cl. CPC A61M 5/31513; A61B 5/1405; A61B 5/153; A61M 2005/3131

(57) ABSTRACT

The invention relates to a device comprising at least a body (1′), a pusher (2′), and a plunger (3′) situated at the end of the pusher for providing sealing between the pusher and the body, the device being characterized in that at least one firstly of the entire inside surface (11′) of the body and secondly of at least a portion of the outside surface (33′) of the plunger (3′) is coated in a hydrophilic substance selected from sodium hyaluronate (HANa), polyvinyl pyrrolidone (PVP) of high molecular weight, polyethylene glycol (PEG) of high molecular weight, and hydroxypropyl methyl cellulose (HPMC).

The invention relates to a device comprising a body, a pusher, and a plunger situated at one end of the pusher for providing sealing between the body and the pusher.

More precisely, the invention relates to such a device having hydrophilic treatment to improve sliding between the plunger and the body.

It may be a syringe, a pump, or any other known device forming a device for delivering a fluid, in particular for medicinal purposes.

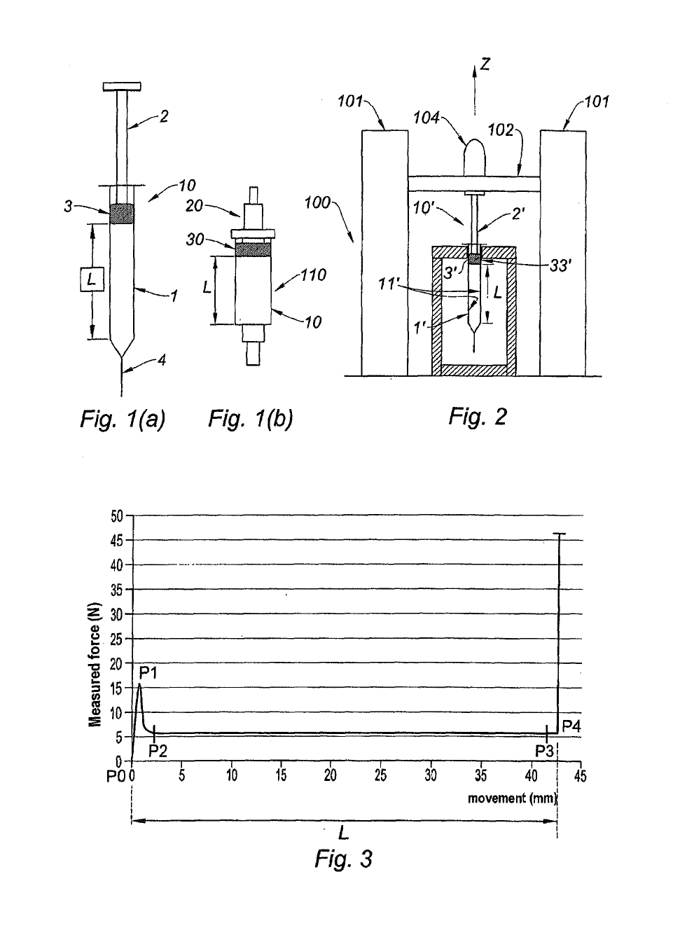

FIG. 1(a) shows a conventional syringe.

The syringe 10 has a syringe body 1, a pusher 2, and a plunger 3 having the function of providing sealing between the syringe body 1 and the pusher 2. For this purpose, the plunger 3 is generally made of elastomer. The plunger 3 is generally mounted at one end of the pusher 2.

The syringe 10 also has a needle 4 arranged at the end of the syringe body to inject the fluid contained in the syringe body 1 into a human or an animal, or on the contrary to extract a sample of blood or other fluid. In other circumstances, the syringe need not have a needle, but may instead be provided with connections for connecting in non-limiting manner with tubes, catheters, or pouches. By way of example, it may be provided with a “Luer” type connection.

The plunger 3 must also present good sliding characteristics.

When a user injects a fluid or takes a sample of blood or other fluid, good sliding makes the syringe easier to use.

For this purpose, the plunger 3 is generally coated in silicone oil.

However, although silicone oils are biocompatible, it is possible they will not be usable in the future. In particular, more and more medicinal fluids are proteins synthesized by biotechnologies. Unfortunately, such proteins, e.g. antibodies, are generally large molecules including hydrophobic blocks that are poorly compatible with the use of silicone oil (referred to below as “silicone”).

FIG. 1(b) shows a pump as conventionally used for injecting a medicinal substance into a human via a natural orifice (mouth, nose), or by injection through the skin.

The pump 110 has a pump body 10, a pusher/piston 20 and a plunger 30 having the function of providing sealing between the syringe body 10 and the pusher 20. Under such circumstances, the plunger 30 belongs to the plug 20 and forms one of its ends.

Like syringes, known pumps are often coated in silicone, generally on the pump body 10.

An object of the invention is thus to propose a device of the above-specified type that does not include silicone and that presents sliding characteristics that are at least substantially similar to those of a silicone-coated device.

Another object of the invention is to propose a device of the above-specified type with sliding characteristics that are improved compared with those of silicone.

In particular, it should be observed that the plunger may stick to the wall of the body of the device when the pusher begins to be set into motion relative to the body.

Under such circumstances, the user thus has a tendency to press harder on the pusher in order to break loose the plunger. As a result, a fluid may be injected into a human or an animal for treatment in a manner that is rough, and possibly painful with certain fluids.

That is why a more particular object of the invention is to provide a device of the above-specified type that provides performance that is comparable, or indeed, in comparison with devices in which the plunger and/or the body is/are coated in silicone, that limits risks of the plunger jamming in the body while the pusher is being set into motion.

Also in particular, it should be observed that during the stroke of the plunger in the body of the device, the sliding of a silicone-coated plunger in the body can also be imperfect and/or may require non-negligible thrust force.

That is why another particular object of the invention is to propose a device that, in comparison with devices in which the plunger and/or the body is/are coated in silicone, presents sliding of the plunger along the body of the device that is comparable, or even better.

To solve at least one of these problems, the invention thus proposes a device comprising at least a body, a pusher, and a plunger situated at the end of the pusher for providing sealing between the pusher and the body, the device being characterized in that at least one firstly of the entire inside surface of the body and secondly of at least a portion of the outside surface of the plunger is coated in a hydrophilic substance selected from sodium hyaluronate (HANa), polyvinyl pyrrolidone (PVP) of high molecular weight, polyethylene glycol (PEG) of high molecular weight, an hydroxypropyl methyl cellulose (HPMC).

The device may also present at least one of the following characteristics, taken singly or in combination:

the entire inside surface of the body and at least a portion of the outside surface of the plunger are coated in the same hydrophilic substance;

the entire outside surface of the plunger is coated in the hydrophilic substance;

the polyvinyl pyrrolidone of high molecular weight is PVP K90;

the polyethylene glycol of high molecular weight is PEG 900,000;

the HPMC is HPMC E4M;

the body is made of glass or of a polymer such as a cyclic olefin copolymer, polyethylene, or polypropylene;

the device is not a syringe;

the device is a pump;

the device is a syringe; and

the plunger is made of elastomer.

The invention can be better understood and other objects, advantages, and characteristics thereof appear more clearly on reading the following description, which is made with reference to the accompanying drawings, in which:

FIG. 2 is a diagram of an experimental set-up for use in characterizing the mechanical properties of a device in accordance with the invention;

FIG. 3 plots a theoretical curve showing the force applied to the pusher of the device as a function of the movement of the pusher along the body, when using the experimental set-up of FIG. 2;

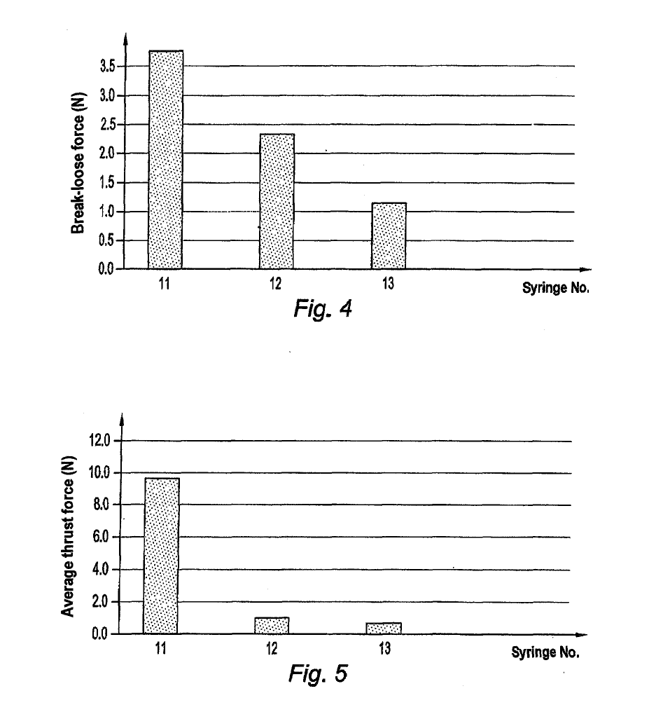

FIG. 4 shows the break-loose force measured using the experimental set-up of FIG. 2 for the plunger of a syringe in accordance with the invention relative to the body of the syringe when the plunger is set into motion in the body of the syringe, with this being shown for various different syringes;

FIG. 5 shows the thrust force measured using the experimental set-up of FIG. 2 that is exerted on the pusher of the syringe while the plunger is moving along the body of the syringe after it has broken loose, with this being shown for the syringes that have their break-loose forces shown in FIG. 4;

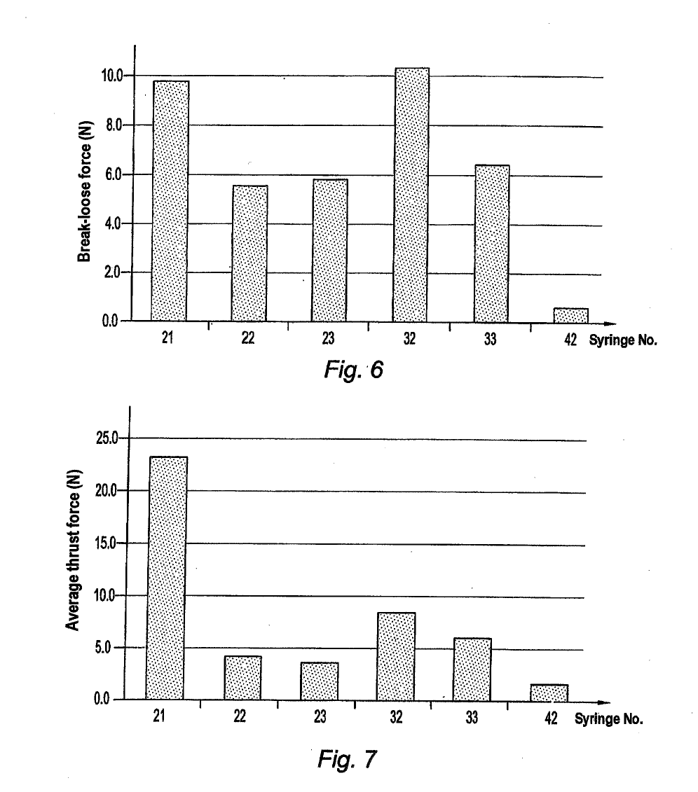

FIG. 6 shows the break-loose force measured using the experimental set-up of FIG. 2 for the syringe plunger relative to the body of the syringe when the plunger is set into motion in the body of the syringe, with this applying to other syringes;

FIG. 7 shows the thrust force measured using the experimental set-up of FIG. 2 as exerted on the pusher of the syringe while the plunger is moving along the body of the syringe after it has broken loose, with this applying to the syringes that have the break-loose forces shown in FIG. 6;

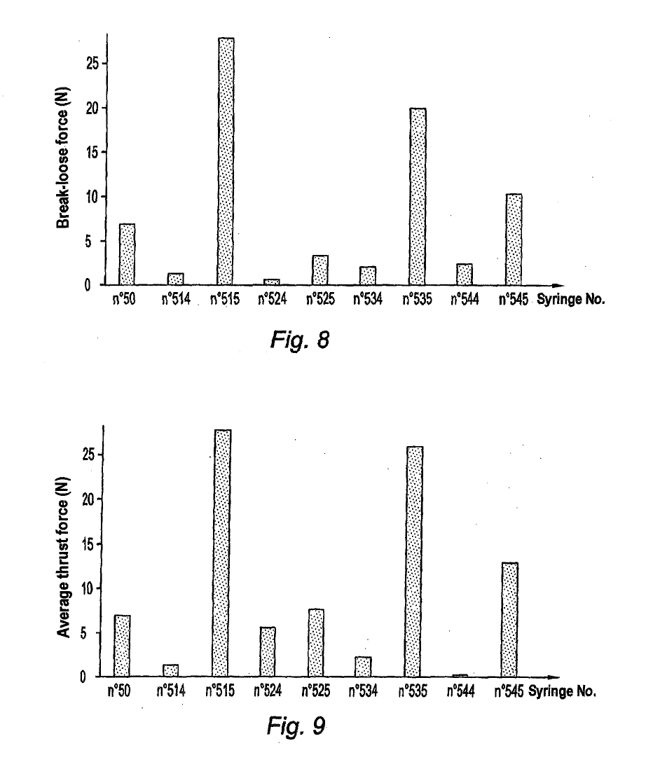

FIG. 8 shows the break-loose force measured using the experimental set-up of FIG. 2, for the syringe plunger relative to the body of the syringe when the plunger is set into motion in the body of the syringe, with this applying to still further syringes; and

FIG. 9 shows the thrust force measured using the experimental set-up of FIG. 2 as exerted on the pusher of the syringe while the plunger is moving along the body of the syringe after it has broken loose, with this applying to the syringes that have the break-loose forces shown in FIG. 8.

FIG. 2 is a diagram of an experimental set-up enabling various devices in accordance with the invention to be tested.

The experimental set-up 100 comprises a dynamometer sold under the trademark MTS, having a stand 101 and a cross-member 102 resting on the stand 101. The cross-member can move along a vertical axis (axis Z) along the stand 101.

The experimental set-up 100 is shown in particular in combination with a syringe, since the various tests described below were performed with such a syringe.

The body 1′ of the syringe is put into place on a carrier 103 that serves to hold the syringe body in place. The pusher 2′ is mounted on the cross-member 102 and can thus be subjected to movement in axial translation (axis Z) in the body 1′ of the syringe 10′ when the cross-member 102 is set into motion along the stand 101. The end of the pusher 2′ is provided with a force sensor 104 that thus serves to determine the force applied to the pusher 2′.

The experimental set-up as shown in FIG. 2 serves to perform compression tests, i.e., when testing a syringe, causing the pusher 2′ to penetrate into the body 1′ of the syringe 10′.

Before testing a device in accordance with the invention on the above-described experimental set-up, the following treatment needs to be performed on the or each portion that is to receive molecules seeking to replace silicone. For a device coated in silicone, it is generally the manufacturer who provides the device already with its silicone coating, so such treatment is then not necessary.

This prior treatment is plasma treatment, performed using an Isytech Plasmatreat RF machine. The portions of the device that are to be treated are placed in an enclosure having electrodes and an inlet for reagent gases such as argon, dinitrogen, or dioxygen. A vacuum is established inside the enclosure (0.1 millibars (mbar)). The plasma is generated by a high frequency generator operating at 13.56 megahertz (MHz), with a maximum power of 600 watts (W).

The plasma treatment improves the wettability of the portions of the syringe that are to be coated by the selected molecule, and thus serves to facilitate attachment of the molecule on the portion in question of the syringe.

Specifically, the molecule is a hydrophilic substance.

As described in detail below, the various molecules tested were selected from:

the family of glycosaminoglycans such as derivatives of hyaluronic acid such as sodium hyaluronate (HANa) or such as heparin;

vinyl polymers such as polyvinyl pyrrolidone (PVP) of high molecular weight (e.g. 1,300,000 grams per mole (g.mol⁻¹); Kollidon® 90F from BASF PVP K90), or polyvinyl pyrrolidone of low molecular weight (e.g. 60,000 g.mol⁻¹; Kollidon® 30 from BASF PVP K30);

hydrosoluble polyethers such as polyethylene glycol (PEG) of high molecular weight (e.g. PEG of 900,000 g.mol⁻¹) or polyethylene glycol (PEG) of low molecular weight (e.g. PEG of 150,000 g.mol⁻¹, also known as PEG 1500), or indeed;

cellulose derivatives such as hydroxypropyl methyl cellulose (HPMC), e.g. HPMC E4M (e.g. known under the trade name Methocel™ E4M from Dow Wolff Cellulosics, or Benecel™ E4M from Ashland), or ethyl cellulose (e.g. Ethocel™ from Dow Wolff Cellulosics, or indeed Surelease® Clear from Colorcon).

A molecule of high molecular weight is a molecule presenting a molecular mass that is greater than or equal to 90,000 g.mol⁻¹, or greater than or equal to 100,000 g.mol⁻¹, or greater than or equal to 200,000 g.mol⁻¹, or indeed greater than or equal to 300,000 g.mol⁻¹. By default, a molecule is considered as presenting low molecular weight when it presents a molecular mass that is strictly less than 90,000 g.mol⁻³.

It should be observed that HANa comes within the category of “high molecular weight” molecules. In contrast, heparin and ethyl cellulose come within the category of “low molecular weight”.

The preparation of the hydrophilic substance takes place in an aqueous solution. The concentration of the hydrophilic substance in the solution may lie in the range 0.05% to 2% and advantageously in the range 0.1% to 2%, in the range 0.2% to 2%, or indeed in the range 1% to 2%.

Going above 2% is found not to be of any use in obtaining better coating. Furthermore, the higher this percentage the greater the cost of production.

The portions of the device that are treated by plasma are then dipped in the solution, allowed to drain, and dried.

As a result of this dipping operation, it can incidentally be deduced that the entire inside surface 11′ of the body 1′ of the syringe and/or where appropriate, the entire outside surface 33′ of the plunger 3′ is/are coated. The entire inside surface 11′ of the body 1′ of the syringe (over length L; cf. FIG. 2) may come into contact with the plunger 3′. Likewise, in the example of FIG. 2, the plunger 3′ presents a shape in which the entire outside surface 33′ is to come into contact with the body 1′ of the syringe.

Nevertheless, the shape of the plunger 3′ may vary from one syringe to another. For example, there are plungers that have one or more portions for coming into contact with the inside surface 11′ of the body 1′ and one or more portions that are set back, which generally do not come into contact with said inside surface 11′ of the body 1′, even in the event of the elastomer forming the plunger 3′ becoming deformed.

The device is then ready to be tested using the experimental set-up described with reference to FIG. 2.

The experimental set-up 100 makes it possible to obtain curves plotting the force measured by the sensor 104 as a function of the movement of the pusher in the body of the device.

The theoretical shape of such a curve is shown in FIG. 3, for a syringe.

Between the point P0 and P1, the movement of the pusher 2′ begins. It should be observed that the force needed to set the pusher into motion increases as a result of friction between the plunger 3′ and the body 1′ of the syringe 10′.

The point P1 corresponds to a local peak in the force measured by the sensor, which peak is associated with the force needed for breaking loose or unjamming the plunger from the wall of the syringe body.

This force is referred to as the break-loose force.

In the context of the invention, it is advantageously desirable to reduce this break-loose force relative to the break-loose force observed using a prior art syringe (plunger coated in silicone).

Between the points P1 and P2, the force decreases as a result of the plunger breaking loose (relaxation associated with breaking loose).

Between the points P2 and P3, the thrust force is monotonic, but not necessarily less than the local force peak associated with the break-loose force. This thrust force corresponds to the force exerted throughout the thrusting of the pusher 2′ along the body 1′ of the syringe 10′, after the stage of breaking the plunger 3′ loose has terminated.

The points P2 and P3 are used for calculating a force referred to as the mean thrust force that is exerted on the pusher between these two points.

The point P4 corresponds to the plunger 3′ coming into abutment against the end of the body 1′ of the syringe 10′.

The measurement comes to an end when the measured force is 90 newtons (N) (not shown in FIG. 3 for reasons of convenience). More precisely, tests are stopped when a force is measured that is equal to 90% of the maximum force that the cell can measure. Specifically, this maximum force is 100 N. This is consistent with the plunger 3′ coming into abutment against the end of the body 1′ of the syringe 10′.

The shape of the curve shown in FIG. 3 is generic. Thus, the same shape would also be obtained for a pump of the kind shown in FIG. 1(b).

Tests 1 to 4 that are described below were performed under the following conditions: travel speed of the cross-member 102 constant and specifically 40 millimeters per minute (mm/min) (typical speed for a syringe); body 1′ of the syringe 10′ filled with pure water; test carried out along the entire length L of the body 1′ of the syringe 10′.

The body 1′ of the syringe 10′ may be made of glass, or of a polymer material such as a cyclic olefin copolymer, polyethylene, or polypropylene. In the context of the tests 1 to 4 described below for a syringe, the body 1′ of the syringe 10′ was made of glass.

The plunger 3′ of the syringe 10′ is generally made of elastomer material. The elastomer used for the plunger 3′ may be selected from polyisoprene, butyl, chlorobutyl, or bromobutyl synthetic rubbers, natural rubber, or mixtures and copolymers of those various materials. Specifically, it was made of chlorobutyl rubber in the tests described below, which tests were performed with syringes.

### TEST 1: Sodium Hyaluronate (HANa)

Three families of syringes with different treatments were tested:

syringe family No. 11: plunger treated with silicone (prior art reference);

syringe family No. 12: syringe body untreated and plunger treated with HANa (invention); and

syringe family No. 13: syringe body and plunger both treated with HANa (invention).

For each syringe family, a plurality of tests were performed in order to obtain averaged data, specifically five tests on five syringes prepared in the same manner.

The data provided in FIGS. 4 and 5 thus comprises, for each syringe family, data as averaged over the five tests. In the specific circumstance of the averaged thrust force, a first average was thus taken between the points P2 and P3 for each test, and then a second average was taken between the various tests.

Below, each syringe family reference is used as a syringe reference.

Furthermore, in order to prepare syringes No. 12 and No. 13, the concentration of HANa in the aqueous solution was 0.2%.

FIG. 4 shows the comparative results obtained for those various syringes concerning the break-loose force.

In FIG. 4, it can be seen that syringes 12 and 13 in accordance with the invention present a break-loose force that is smaller than the break-loose force measured for the reference syringe (silicone-coated plunger).

In other words, these syringes limit the risk of, the plunger 3′ jamming while the plunger is being set into motion in the body 1′ of the syringe.

Furthermore, it matters little whether the HANa treatment is performed on the plunger 3′ alone or both on the body 1′ of the syringe 10′ and on the plunger 3′.

Nevertheless, it may be observed that in a syringe where both the plunger 3′ and the body 1′ are coated in HANa (syringe No. 13), the break-loose force was smaller than for a syringe in which only the plunger 3′ was coated in HANa (syringe No. 12).

In summary, the solution requiring the smallest break-loose force consists in coating both the plunger 3′ and the body 1′ of the syringe 10′ with HANa.

FIG. 5 shows the comparative results obtained for the syringes No. 11 (reference), No. 12, and No. 13 concerning the average thrust force exerted by the pusher 2′ in its movement along the body 1′ after the plunger 3′ had broken loose.

Once more, there can be seen a significant reduction in the average thrust force exerted by the pusher 2′ as it moves along the body 1′ for the syringes No. 12 and No. 13 in accordance with the invention compared with that measured with the reference syringe (silicone-coated plunger).

Nevertheless, it may be observed that in a syringe where both the plunger 3′ and the body 1′ are coated in HANa (syringe No. 13), the mean thrust force is smaller than that obtained with a syringe having a silicone-treated plunger and even smaller than that obtained with a syringe in which only the plunger 4′ was coated in HANa (syringe No. 12).

The HANa-coated syringes of the invention thus present sliding characteristics that are improved compared with those in which the plunger is silicone coated.

Nevertheless, the syringe No. 13 is a solution that is particularly advantageous both in terms of break-loose force and in terms of average thrust force.

TEST 2: Polyethylene Glycol, 900,000 g/mol (PEG 900,000)

Three syringe families with different treatments were tested:

syringe family No. 21: plunger treated with silicone (prior art reference);

syringe family No. 22: syringe body untreated and plunger treated with PEG 900,000 (invention); and

syringe family No. 23: syringe body and plunger both treated with PEG 900,000 (invention).

For each syringe family, a plurality of tests were performed in order to obtain averaged data, specifically ten tests were performed with ten syringes prepared in the same manner.

Below, each family reference is used as a syringe reference.

In order to prepare the syringes No. 22 and No. 23, the concentration of PEG in the aqueous solution was 0.2%.

The results of these tests are shown in FIG. 6 for break-loose force and in FIG. 7 for average thrust force.

It can be seen that syringe No. 22 presents a break-loose force smaller than that obtained with the reference syringe (silicone-coated plunger). It can also be seen that the average thrust force is much lower with this syringe No. 22 than that which was obtained using the reference syringe.

Similar remarks can be made comparing syringe No. 23 with the reference syringe.

Furthermore, when syringes No. 22 and No. 23 in accordance with the invention are compared with each other, it can be seen that they provide performance that is comparable, both in terms of break-loose force and in terms of average thrust force.

### TEST 3: Polyvinyl Pyrrolidone K90 (PVP K90)

Three syringe families with different treatments were tested:

syringe family No. 21: plunger treated with silicone (prior art reference):

syringe family No. 32: syringe body untreated and plunger treated with PVP K90 (invention); and

syringe family No. 33: syringe body and plunger both treated with PVP K90 (invention).

For each syringe family, a plurality of tests were performed in order to obtain averaged data, specifically ten tests were performed with ten syringes prepared in the same manner.

Below, each family reference is used as a syringe reference.

In order to prepare the syringes No. 32 and No. 33, the concentration of PVP K90 in the aqueous solution was 0.2%.

The results of those tests are given in FIG. 6 for the break-loose force and in FIG. 7 for the average thrust force.

It can be seen that the syringe No. 32 presents a break-loose force similar to that obtained with the reference syringe (silicone-coated plunger). It can also be seen that the average thrust force obtained with the syringe No. 32 is much lower than that obtained with the reference syringe.

It can also be seen that syringe No. 33 presents a break-loose force that is much lower than that obtained with the reference syringe. Furthermore, it can be seen that the average thrust force is also much lower than that obtained with the reference syringe.

When the syringes No. 32 and No. 33 in accordance with the invention are compared with each other, a syringe having both the plunger and the body coated in PVP K90 is more advantageous, both in terms of break-loose force and in terms of average thrust force.

It should be observed that test 2 and test 3 were performed at the same time, which explains why the selected reference (syringe No. 21) is the same for both of these tests.

### TEST 4: Hydroxylpropyl Methyl Cellulose (HPMC E4M)

The inventors have been able to test a syringe with coating either on the plunger alone, or on both the plunger and the body of the syringe using HPMC E4M. In those tests, the concentration of HPMC E4M in the aqueous solution was 0.2%. These tests show performance similar to that of a reference syringe (silicone-coated plunger) concerning the break-loose force, and performance that is improved concerning mean thrust force.

These tests were associated with another test in which only the plunger was treated with HPMC E4M, with the concentration of HPMC E4M in the aqueous solution then being 1%.

Two syringe families with different treatments were tested:

syringe family No. 21: plunger treated with silicone (prior art reference); and

syringe family No. 42: syringe body untreated and plunger treated with HPMC E4M (invention).

For each syringe family, a plurality of tests were performed in order to obtain averaged data, specifically 20 tests with 20 syringes prepared in the same manner.

Below, each syringe family reference is used as a syringe reference.

For preparing syringes No. 42, the concentration of HPMC E4M in the aqueous solution was 1%, as mentioned above.

The results of these tests are shown in FIG. 6 for break-loose force and in FIG. 7 for average thrust force.

It can be seen that syringe No. 42 presents a break-loose force that is smaller than that obtained with the reference syringe (syringe No. 21). It can also be seen that the mean thrust force is much smaller for this syringe No. 42 than the force obtained with the reference syringe.

A syringe having its body treated with HANa but a plunger that was not treated has not been tested since that constitutes a solution intermediate between the two extreme solutions corresponding to the syringes No. 12 (only the plunger 3′ treated) and No. 13 (both the body 1′ and the plunger 3′ treated). Coating only the syringe body requires coating on a larger area than coating only the plunger, but involves coating an area that is less than coating both the syringe body and the plunger.

Given the results of the tests shown in FIGS. 4 and 5, such a syringe would present the same advantages compared with the reference syringe (silicone-coated plunger), as presented by the syringes No. 12 and No. 13.

Similar remarks may be made when HANa is replaced by PEG 900,000, by PVP K90, or by HPMC E4M.

Finally, among the various molecules that the inventors have identified, it appears that the solution that is the most interesting, in terms of performance compared with the prior art, consists in coating both the plunger and the body of the syringe with HANa. That molecule makes it possible to reduce the break-loose force and the average thrust force in the most significant manner for an identical concentration in the aqueous solution (comparison possible for a concentration of 0.2% in tests 1 to 3, and thus ignoring the additional test in test 4).

The additional test (syringe No. 42) performed with HPMC E4M shows that that substance also makes it possible to solve the problem posed by the invention and, by increasing the concentration of the hydrophilic substance in the aqueous solution (going from 0.2% to 1%), it becomes possible to have an impact on improving the performance of the syringe (decreasing the break-loose force; decreasing the average thrust force).

Other tests (tests 5.1 to 5.4) have been performed with syringes.

Unless specified specifically to the contrary below, the test conditions and the syringes were the same as those described above.

For these other tests, the speed of the cross-member 102 was set at 100 mm/min.

Furthermore, the body 1′ of the syringe 10′ was made of glass.

The purpose of these tests was to show that certain molecules are not advantageous for the intended object.

In all of these tests, syringe family No. 50 for which no treatment was performed is used as a reference (negative control). As is well known to the person skilled in the art, it thus presents sliding properties that are less good than a syringe that has been treated with silicone, where such a syringe was used as a reference (positive control) in tests 1 to 4. In other words, when a syringe presents sliding properties that are not as good as the reference syringe No. 50, then the person skilled in the art can logically expect its sliding properties to be likewise less advantageous than those of a silicone-coated syringe.

For these other tests, a plurality of tests were performed in order to obtain averaged data, specifically 20 tests with 20 syringes prepared in the same manner.

### TEST 5.1: Within the Family of Glycosaminoglycans; Comparison Between HANa and Heparin

Syringe family No. 514: plunger untreated and syringe body treated with HANa (invention); and

syringe family No. 515: plunger untreated and syringe body treated with heparin (not invention).

In order to prepare the syringes No. 514 and No. 515, the concentration of HANa in the aqueous solution was 0.1%.

TEST 5.2: within the family of (hydrosoluble) vinyl polymers; comparison between PVP K90 and PVP K30

Syringe family No. 524: plunger untreated and syringe body treated with PVP K90 (invention); and

syringe family No. 525: plunger untreated and syringe body treated with PVP K30 (not invention).

For preparing syringes No. 524 and No. 525, the concentration of PVP, K90 or K30 as appropriate, in the aqueous solution was 1%.

### TEST 5.3: Within the Family of Hydrosoluble Polyethers; Comparison Between PEG 900,000 and PEG 1500

Syringe family No. 534: plunger untreated and syringe body treated with PEG 900,000 (invention); and

syringe family No. 535: plunger untreated and syringe body treated with PEG 1500 (not invention).

For preparing syringes No. 534 and No. 535, the concentration of PEG, 900,000 or 1500 as appropriate, in the aqueous solution was 1%.

### TEST 5.4: Within the Family of Cellulose Derivatives; Comparison Between HPMC E4M and Ethyl Cellulose (EC)

Syringe family No. 544: plunger untreated and syringe body treated with HPMC E4M (invention); and

syringe family No. 545: plunger untreated and syringe body treated with ethyl cellulose or EC (not invention).

For preparing syringes No. 544 and No. 545, the concentration of cellulose derivative in the aqueous solution was 1%.

The results obtained from these various tests are shown in FIG. 8 concerning break-loose force, and in FIG. 9 concerning average thrust force.

It may be observed that heparin (No. 515), PVP K30 (No. 525), PEG 1500 (No. 535), and EC (No. 545) do not enable advantageous sliding characteristics to be obtained, whether in terms of break-loose force or in terms of mean thrust force along the syringe body.

For the other syringe families, namely HANa (No. 514), PVP K90 (No. 524), PEG 900,000 (No. 5234), and HPMC E4M (No. 544), the results obtained match those shown under the conditions of tests 1 to 4, but for plunger travel speeds relative to the pump body that are faster (100 mm/min for tests 5.1 to 5.4; 40 mm/min for tests 1 to 4).

This shows that the speed at which the plunger 3′ is pushed does not lead to a qualitative change in the results obtained.

Furthermore, it should be observed that for a pump, such as the pump shown in FIG. 1(b), the travel speed of the plunger relative to the body is generally closer to 100 mm/min than to 40 mm/min (since assistance is generally provided in propelling the fluid that is to be dispensed, such as using air under pressure). As a result, the results obtained for a syringe are transposable to the circumstance of a pump.

The description above relates to applications relating to a syringe and to a pump.

Nevertheless, the invention applies more generally to any device having at least a body, a pusher, and a plunger situated at the end of the pusher for providing sealing between the pusher and the body and characterized in that at least one firstly of the entire inside surface of the body and secondly of at least part of the outside surface of the plunger is coated in a hydrophilic substance selected from sodium hyaluronate (HANa), polyvinyl pyrrolidone (PVP) of high molecular weight, polyethylene glycol (PEG) of high molecular weight, and hydroxypropyl methyl cellulose (HPMC).

The device is a device for diffusing a fluid, in particular for medicinal purposes.

In particular, the syringe may be any device comprising at least a body, a pusher, and a plunger situated at the end of the pusher for providing sealing between the pusher and the body, with the exception of a syringe, the device being characterized in that at least one firstly of the entire inside surface of the body and secondly of at least a portion of the outside surface of the plunger is coated in a hydrophilic substance selected from sodium hyaluronate (HANa), polyvinyl pyrrolidone (PVP) of high molecular weight, polyethylene glycol (PEG) of high molecular weight, and hydroxypropyl methyl cellulose (HPMC).

In a variant, the device may be a syringe.

1. A device comprising at least a body (1′), a pusher (2′), and a plunger (3′) situated at the end of the pusher for providing sealing between the pusher and the body, the device being characterized in that at least one firstly of the entire inside surface (11′) of the body and secondly of at least a portion of the outside surface (33′) of the plunger (3′) is coated in a hydrophilic substance selected from sodium hyaluronate (HANa), polyvinyl pyrrolidone (PVP) of high molecular weight, polyethylene glycol (PEG) of high molecular weight, and hydroxypropyl methyl cellulose (HPMC).

2. A device according to claim 1, characterized in that the entire inside surface (11′) of the body (1′) and at least a portion of the outside surface (33′) of the plunger (3′) are coated in the same hydrophilic substance.

3. A device according to claim 1, characterized in that the entire outside surface (33′) of the plunger (3′) is coated in the hydrophilic substance.

4. A device according to claim 1, characterized in that the polyvinyl pyrrolidone of high molecular weight is PVP K90.

5. A device according to claim 1, characterized in that the polyethylene glycol of high molecular weight is PEG 900,000.

6. A device according to claim 1, characterized in that the HMPC is HMPC E4M.

7. A device according to claim 1, wherein the body (1′) is made of glass or of a polymer such as a cyclic olefin copolymer, polyethylene, or polypropylene.

8. A device according to claim 1, with the exception of a syringe.

9. A device according to claim 1, characterized in that it is a pump.

10. A device according to claim 1, characterized in that it is a syringe.

11. A device according to claim 1, characterized in that the plunger (3′) is made of elastomer.
